(19) United States
(12) Patent Application Publication
(10) Pub. No.: US 2004/0133941 A1
(43) Pub. Date: Jul. 8, 2004

(54) Methods for identifying genes regulating desired cell phenotypes

(76) Inventors: Benjamin A. Bowen, Berkeley, CA (US); Edward A. Deakin, Sheffield (GB); Neil Goldsmith, Oxford (GB); Christian Haudenschild, Oakland, CA (US); David R. Houck, Chapel Hill, NC (US); James B. McAlpine, Bolton, MA (US); Soren V.S. Nielsen, Allerod (DK); Christopher Pazoles, Westboro, MA (US); Margaret E. Spencer, Sheffield (GB); Angela M. Stafford, Castleton (GB)

Correspondence Address: Carol A. Stratford, Foley & Lardner LLP, Three Palo Alto Square, 3000 El Camino Real Ste 100, Palo Alto, CA, 94306-2121, US

(21) Appl. No.: 10/785,744

(22) Filed: Feb. 23, 2004

Related U.S. Application Data
(62) Division of application No. 10/056,479, filed on Jan. 24, 2002.
(60) Provisional application No. 60/263,807, filed on Jan. 24, 2001.

(51) Int. Cl. A01H 1/00; C12N 15/82; C12Q 1/68

(52) U.S. Cl. 800/278; 435/6

(57) ABSTRACT

The invention features a method for identifying a gene associated with a desired phenotype. This method includes the steps of: (a) providing a plurality of cell cultures that include plant, animal, or fungal cells capable of exhibiting a desired phenotype; (b) contacting each of at least a subset of said cells with a stimulus that (i) induces said cells to exhibit the phenotype, or (ii) does not induce said cell cultures to exhibit the phenotype; (c) determining the presence of the phenotype in the cell cultures of step (b); and (d) identifying a gene having increased expression in response to stimuli that induce the phenotype but do not have increased expression in response to stimuli that do not induce the phenotype.

### CROSS-REFERENCE TO RELATED APPLICATIONS

This application claims benefit from co-pending U.S. Provisional Application 60/263,807 (filed Jan. 24, 2001), hereby incorporated by reference.

### BACKGROUND OF THE INVENTION

The invention relates to the field of gene identification.

Cultured cells respond differently to different conditions. For example, continuous high light conditions can induce greening and plastid differentiation in plant cells in culture, while the same cells exhibit a stress response when treated with methyl jasmonate. Generally, the cellular response involves the alteration in the level of expression of one or more genes.

When presented with a pathogen (or a signal that mimics the presence of a pathogen), plant cells often respond by producing antipathogenic compounds as part of a defense mechanism. We and others have used this host response to pathogens as a means to identify naturally-occurring antipathogenic compounds. For example, PCT Publication No. WO01/25197 describes antifungal compounds identified from plant cultures treated in sequence with a methylation inhibitor and an elicitor.

Antipathogenic compounds are not the only compounds made by plants in response to different external stimuli and having commercial potential. Many plant-derived compounds have been shown to be useful as pharmaceuticals for a wide variety of therapies including treatment of cancer, pain, cardiovascular disease, depression, etc. In addition, they are useful as pesticides (e.g., insecticides, microbiocides, molluscicides, and arachnicides). They are also widely used as aromatics, flavoring agents, antioxidants, and dyes or other coloring agents. By altering culture conditions, it may be possible to increase the levels of such commercially-relevant plant-derived compounds, or induce the production of a wider variety of compounds. Applications of such methods include (1) discovery and identification of novel biologically active compounds in extract mixtures (2) more economic industrial-scale production and (3) discovery of the critical genes involved in the biosynthesis of the commercially relevant compounds.

### SUMMARY OF THE INVENTION

The production of a plant compound in response to an external stimulus is likely associated with an alteration in expression of genes encoding transcription factors that coordinate the overall response to the stimulus as well as genes encoding proteins (e.g., an enzyme) involved in the production of that compound. Identification of the stimuli and genes allows for the production of transgenic plant cells that overexpress them and are thus likely to produce increased amounts of the compound. Because many plant compounds have commercial value, increased production of such plant compounds is desirable. Using prior art methods, it is not trivial to identify either of the foregoing classes of genes. In response to any one stimulus, a plant cell may exhibit altered gene expression for tens or hundreds of transcripts, many of which are not directly associated with the increased production of the desired compound. We have discovered a method for identifying the relevant transcripts from among those that are altered. This method is suitable for identifying genes, from any cell, that exhibit altered expression and whose expression also results in an observable phenotype in response to an external stimulus.

Accordingly, in one aspect, the invention features a method for identifying a gene associated with a desired phenotype. This method includes the steps of: (a) providing a plurality of cell cultures that include plant, animal, or fungal cells capable of exhibiting a desired phenotype; (b) contacting each of at least a subset of said cells with a stimulus that (i) induces said cells to exhibit the phenotype, or (ii) does not induce said cell cultures to exhibit the phenotype; (c) determining the presence of the phenotype in the cell cultures of step (b); and (d) identifying a gene having increased expression in response to stimuli that induce the phenotype but do not have increased expression in response to stimuli that do not induce the phenotype. Genes identified by this method have a high likelihood of being associated with the desired phenotype. The function of these genes can then be confirmed by classical methods of gene identification (e.g., nucleotide sequencing) and manipulation (e.g., transformation) well known to those skilled in the arts and enumerated later.

By “phenotype” is meant an observable or measurable cell or cell culture characteristic. Suitable phenotypes include, but are not limited to, production of a protein or compound (e.g., a secondary metabolite), ability to proliferate, ability to grow on a particular substitute such as soft agar, ability to withstand heat, high salinity, desiccation, or freezing and thawing, color, size, and ability to utilize uncommon energy sources.

The phenotype may be, for example, accumulation of isoprene-containing compounds such as terpenes (e.g., monoterpenes, diterpenes, sesquiterpenes), or accumulation of catechins (e.g., epigallocatechin gallate, epicatechin gallate, epigallocatechin, gallocatechin). The plant cells can include any plant cells capable of being cultured. Exemplary plant cells include Ajuga reptans cells, Taxus baccata cells, cells of a species of the family Crassulaceae (e.g., Crassula fascicularis, C. dejecta, C. barkleyi, C. acinaciformis, Sempervivum tectorum) and cells of the family Polygonaceae (e.g., Fallopia convolvulus, Rumex obtusifolia, or R. sagittatus).

The gene associated with the desired phenotype can be one encoding an enzyme (e.g., one in a biosynthetic pathway for the production of a terpene or a catechin). The phenotype can be induced by an appropriate stimulus (such as methyl jasmonate, zeatin, 24-epibrassinolide, or 1-aminocyclopropane-1-carboxylic acid, or a preparation from Candida albicans).

The invention also features a method for producing a substantially pure catechin (e.g., epigallocatechin gallate, epigallocatechin, epicatechin gallate, or gallocatechin). In one method, the catechin is purified from plant cells of the genus Crassula. The plant cells may be, for example, in the form of a plant cell culture or in the form of a plant or plant component (e.g., a leaf, shoot, root, or seed). In another method, the catechin is purified from a suspension culture of plant cells of the genus Fallopia. In still another method, the catechin is purified from a suspension culture of plant cells of the genus Rumex.

The invention also features a method for identifying a compound that increases production of a catechin in a plant cell. This method includes the steps of: a) providing plant cells capable of producing a catechin; b) contacting the plant cells with a candidate compound or preparation; and c) determining the levels of the catechin in the plant cells, wherein an increase in the levels of the catechin identifies the candidate compound or preparation as a compound or preparation that increases production of the catechin.

In a related aspect, the invention features a method for identifying a protein that increases production of a catechin in a plant cell. This method includes the steps of: a) providing plant cells capable of producing a catechin; b) transgenically expressing in the plant cells a nucleic acid encoding a candidate protein; and c) determining the levels of the catechin in the plant cells, wherein an increase in the levels of the catechin identifies the candidate protein as a protein that increases production of said catechin.

By “external stimulus” or “culture condition” is meant the environment in which a cultured cell is placed and to which it responds.

By “a catechin” is meant compounds selected from a group consisting of catechin itself, stereoisomers of catechin, or naturally occurring derivatives of catechin or stereoisomers of catechin, including, for example, epigallocatechin gallate, epicatechin gallate, epigallocatechin, gallocatechin, and gallocatechin gallate.

By “plurality” is meant two or more, preferably three, four, five, six, seven, eight, or more.

As used herein, measurement of “gene expression” is not limited to determination of the level of mRNA, but also encompasses measurement of the relative level of protein, or enzymatic activity resulting from downstream translation of the mRNA.

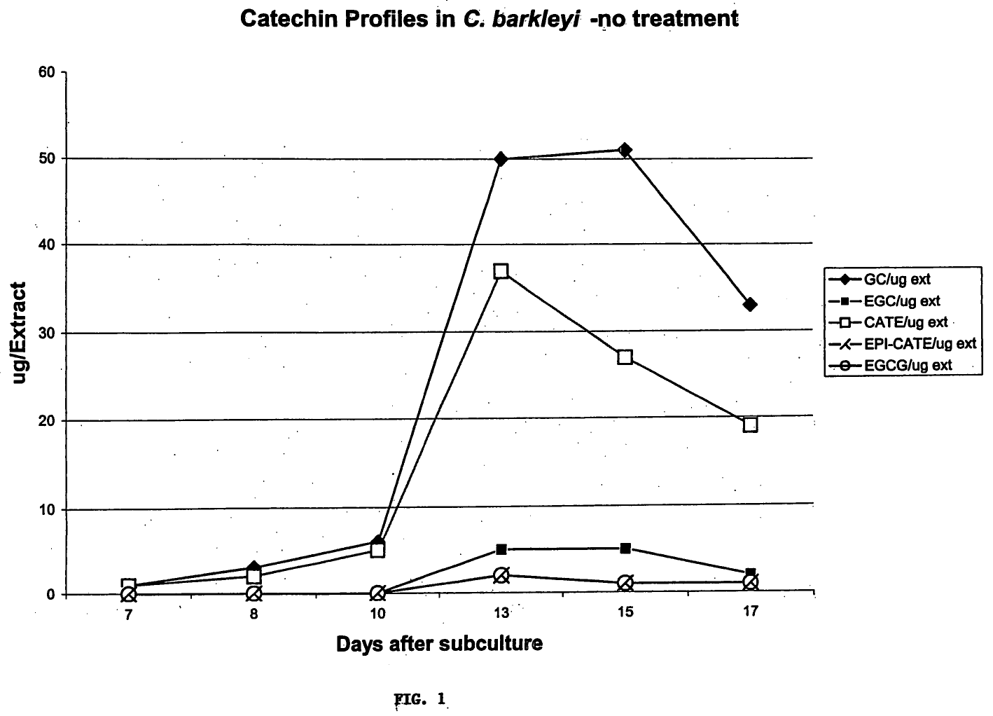

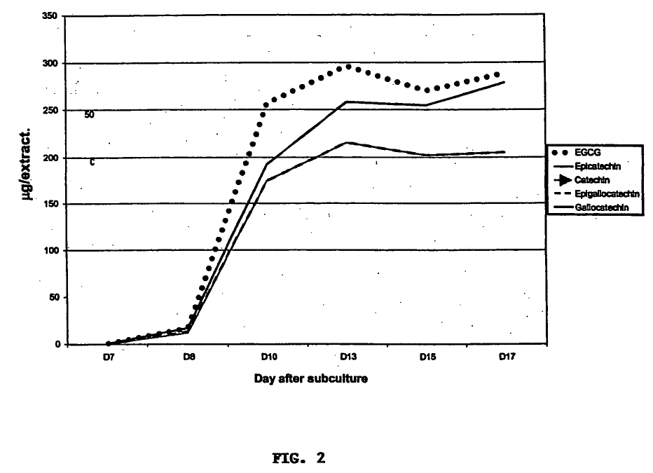

FIG. 1 is a schematic illustration showing the profile of catechin accumulation in Crassula barkleyi cells cultured without any inducing agent on day 7.

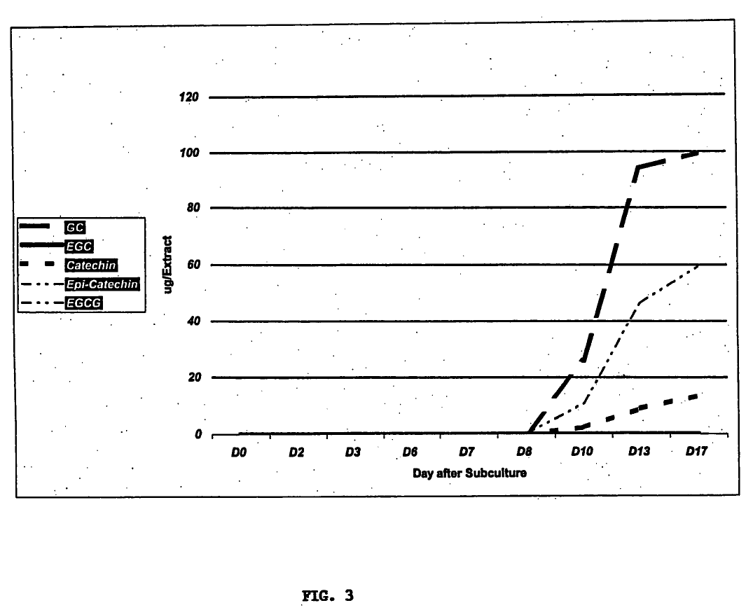

FIG. 2 is a schematic illustration showing the effect of day 7 methyl jasmonate treatment and DL-phenylalanine on catechin accumulation in cultured Crassula barkleyi cells.

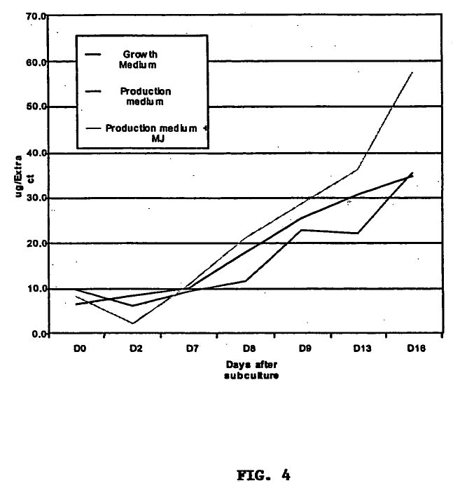

FIG. 3 is a schematic illustration showing the effect of day 7 methyl jasmonate treatment on catechin accumulation in cultured Crassula dejecta cells.

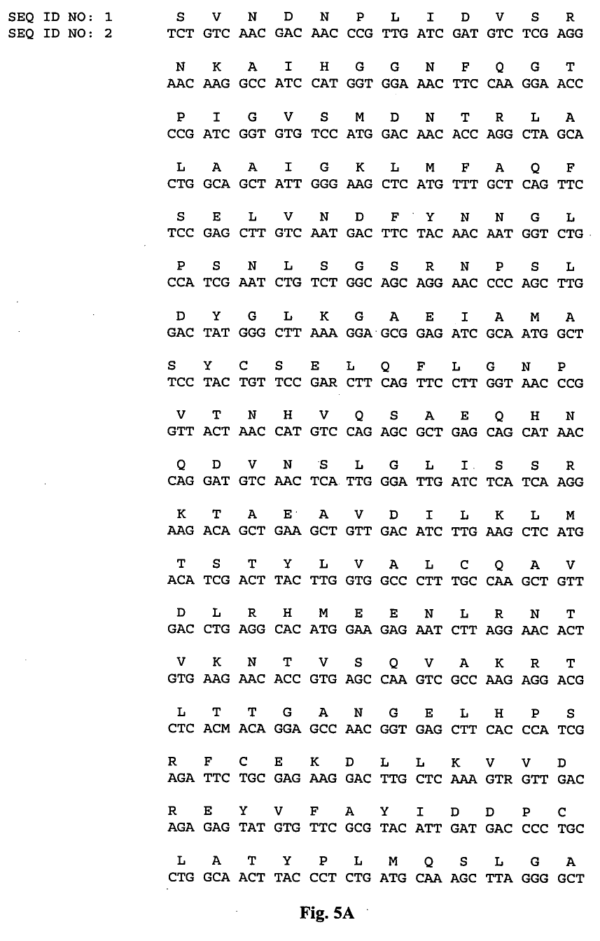

FIG. 4 is a schematic illustration showing the effect of day 7 methyl jasmonate treatment or media choice on catechin accumulation in cultured Crassula acinaformis cells.

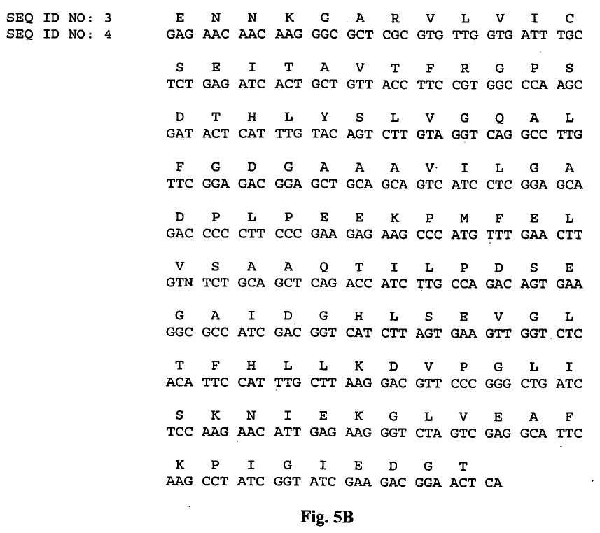

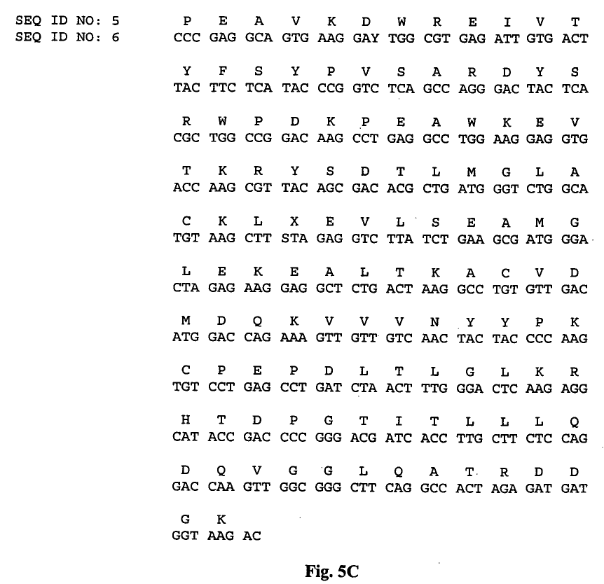

FIG. 5A is a schematic illustration showing the amino acid and nucleic acid sequence of L-phenylalanine ammonia lyase (PAL; SEQ ID NOs: 1 and 2, respectively) from C. barkleyi.

FIG. 5B is a schematic illustration showing the amino acid and nucleic acid sequence of chalcone synthase (CHS; SEQ ID NOs: 3 and 4, respectively) from C. barkleyi.

FIG. 5C is a schematic illustration showing the amino acid and nucleic acid sequence of flavanone-3β-hydroxylase (F3-OH; SEQ ID NOs: 5 and 6, respectively) from C. barkleyi.

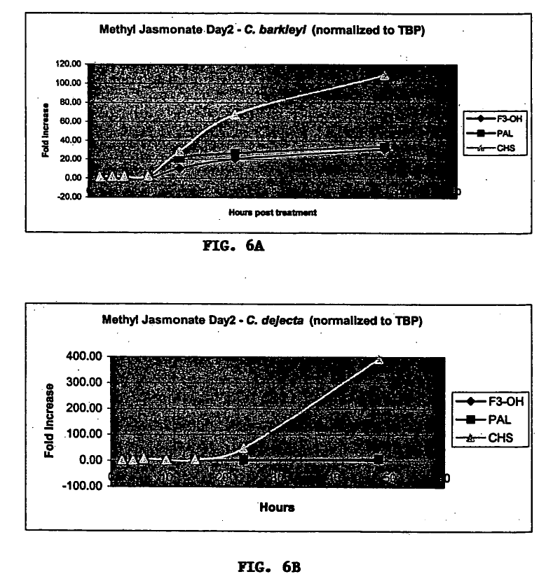

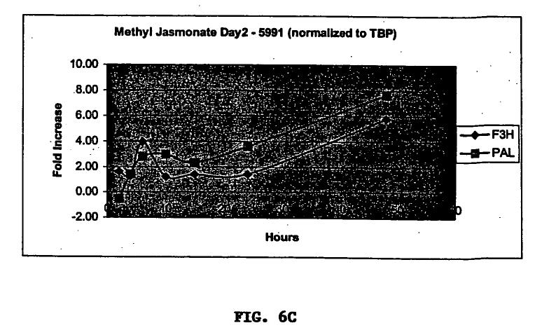

FIG. 6A is a schematic illustration showing a quantitative RT-PCR survey of PAL, CHS, and F3-OH in C. barkleyi cells following treatment with methyl jasmonate at day 2.

FIGS. 6B and 6C are schematic illustrations showing a quantitative RT-PCR survey of PAL, CHS, and F3-OH in C. dejecta cells following treatment with methyl jasmonate at day 2.

FIG. 7 shows the sequences of oligonucleotides used in SYBR green assays.

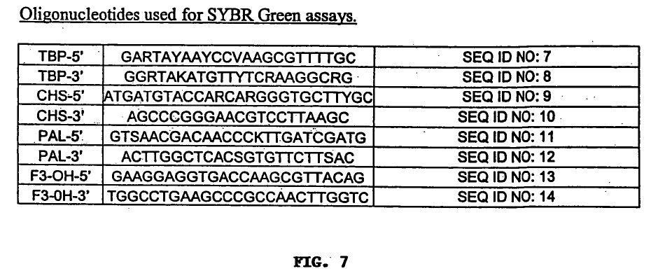

FIG. 8 is a schematic illustration showing catechin accumulation profiles in Sempervivum tectorum in B49 media with and without treatment with methyl jasmonate (MJ) on day 7 after subculture.

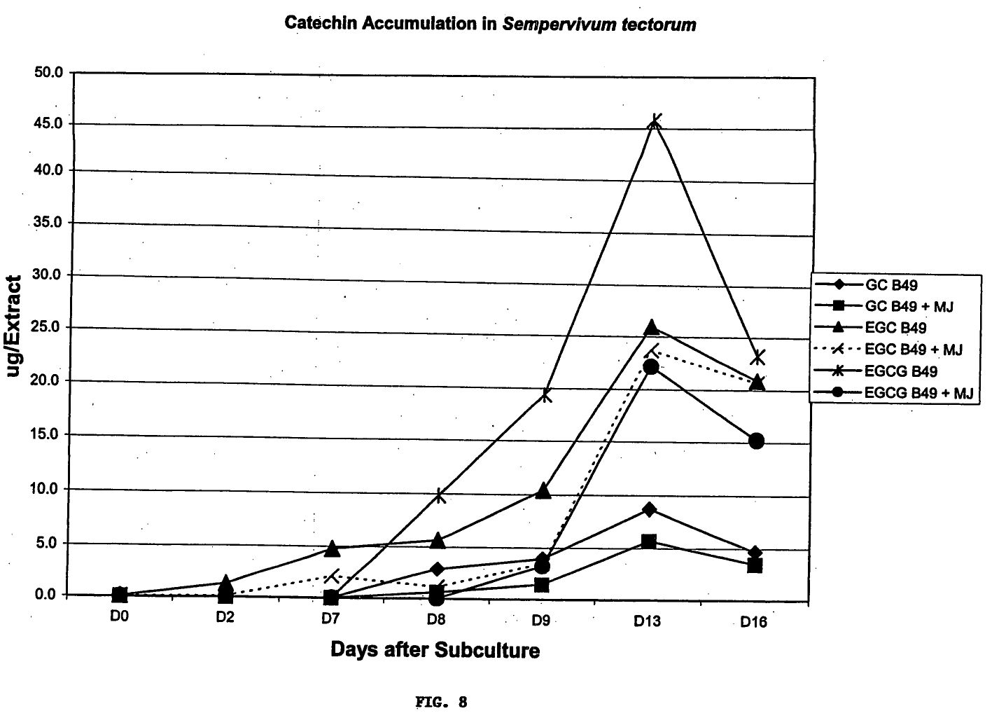

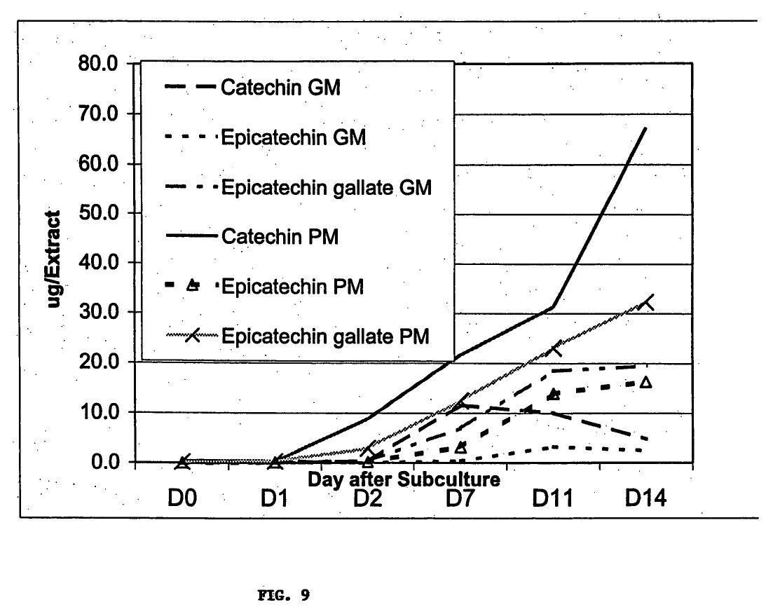

FIG. 9 is a schematic illustration showing the effect of medium on the production of catechins in Fallopia convolvulus suspension cell cultures.

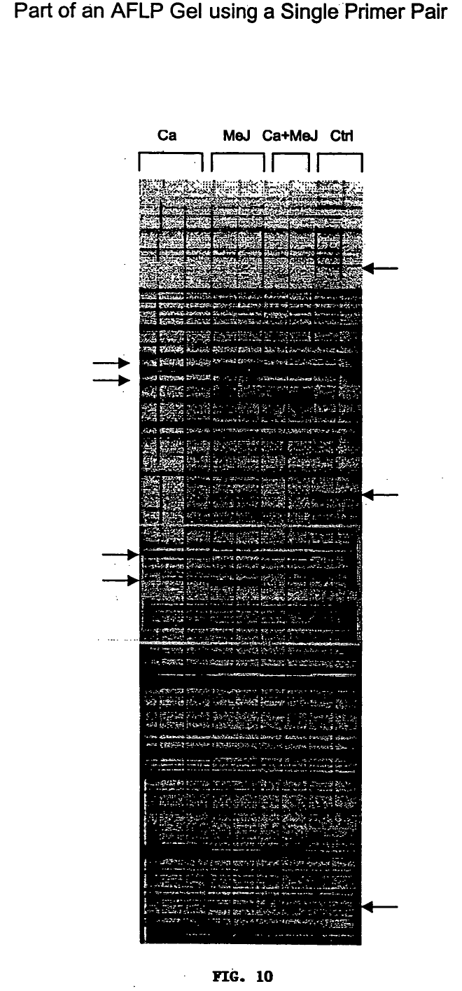

FIG. 10 is a schematic illustration showing part of and AFLP gel produced using a single primer pair. The arrows denote differentially amplified bands.

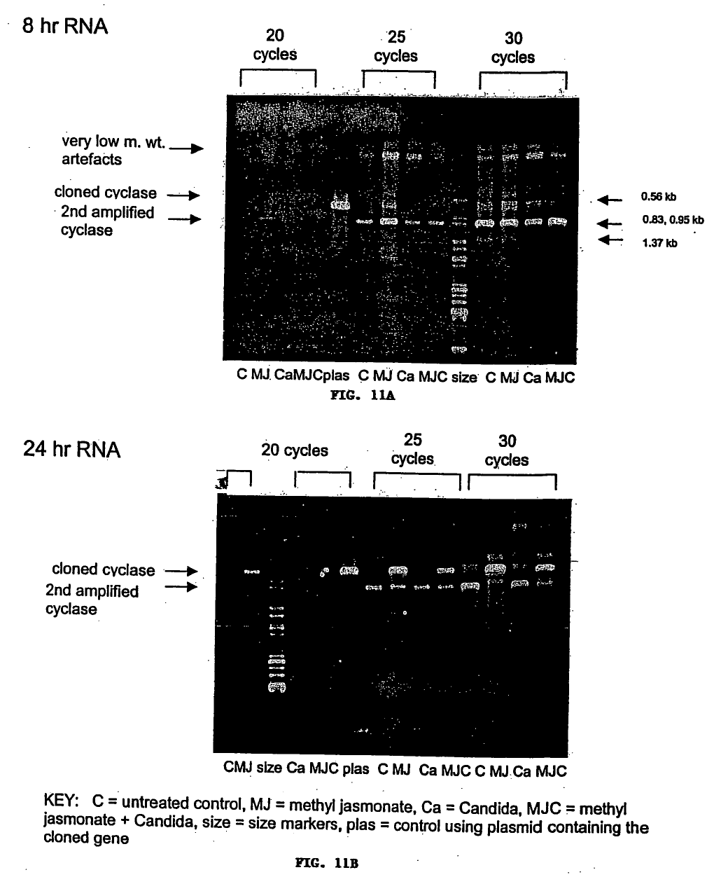

FIGS. 11A and 11B are photographs of agarose gels depicting the results of PCR of Ajuga reptans RNA with primers based on a cyclase identified by RACE.

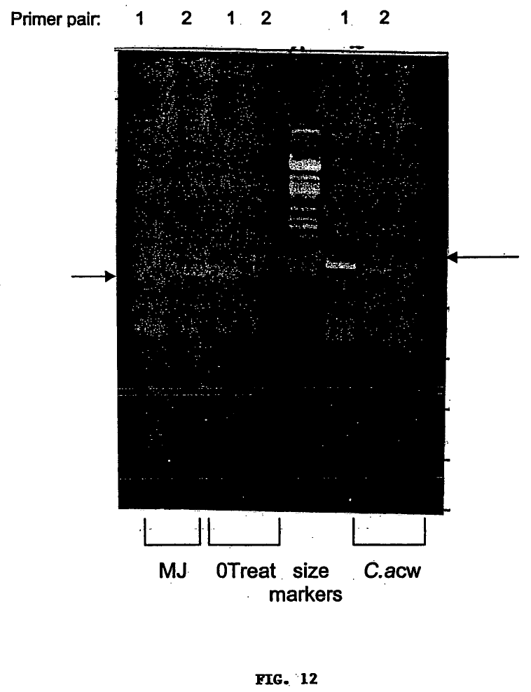

FIG. 12 is a photograph of an agarose gel depicting PCR amplification of cyclases following various treatments of cultured Ajuga reptans cells.

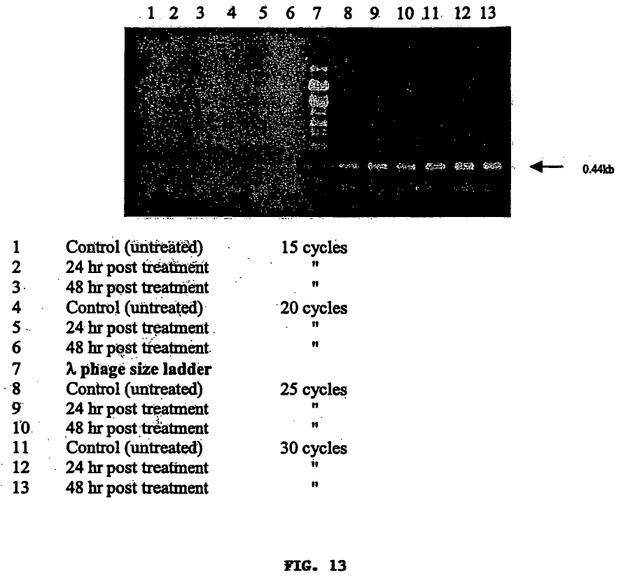

FIG. 13 is a photograph of an agarose gel showing expression of taxadiene synthase from untreated and methyl jasmonate treated Taxus baccata callus cultures using taxadiene synthase PCR probes.

Other features and advantages of the invention will be apparent from the following description of embodiments thereof, and from the claims.

### DETAILED DESCRIPTION

We have developed a method by which genes that may regulate or determine a desired cell culture phenotype can be identified. The method is based on the prediction that while different external stimuli will each induce cultured cells to have different gene expression profiles, those stimuli that induce a desired phenotype (e.g., production of a particular secondary metabolite, cell proliferation, etc.) will have genes in common, the induction of altered expression of a subset of genes responsible for that phenotype. By identifying genes having altered expression under culture conditions that induce a desired phenotype but not having altered expression under conditions that do not induce that phenotype, we can rapidly identify genes that coordinate the response to the stimulus (e.g., transcription factors), as well as those that are part of the response pathway (e.g., biosynthetic enzymes for the production of secondary metabolites).

There are numerous techniques known in the art for identifying mRNA of differentially expressed genes, including use of gene chips or differential display (Table 1). Any of these methods is applicable for use in the present invention. If desired, mRNAs encoding candidate classes of proteins (e.g., certain classes of transcription factors, biosynthetic enzymes, etc.) can be screened using degenerate RT-PCR The method is also amenable to whole-genome screening e.g., array probing to identify differentially-expressed genes that would not a priori have been associated with the observed phenotype. In addition to measuring mRNA levels, one can also measure altered gene expression by measuring relative protein levels or protein activity.

TABLE 1

| | | |
|---|---|---|
| Product | Measurement | Description |

MRNA

| | | |
|---|---|---|
| Overall display methods | Differential display | PCR on total mRNA (cDNA) using arbitrary |
| | | primers - display on sizing gel |
| | AFLP | PCR amplification of restriction fragments - |
| | | display on sizing gel |
| | Array probing | Hybridizing labeled mRNA/cDNA to an |
| | | immobilized array of ‘total’ transcripts. |
| | Megasort ® | FACS sorting of transcripts after probing with |
| | | ‘total’ RNA loaded micro breads |
| | MPSS ® | Mass parallel sequencing of transcript 3′ ends |
| | | using cDNA clones loaded on micro beads |
| | SAGE | Mass sequencing of transcript 3′ ends |
| | | (10-14 bases) |
| Detection of specific | Probing | Probing with labeled synthetic RNA/DNA. |
| transcripts | | Often oligonucleotide, often degenerate mixtures. |
| | | Requires some prior knowledge. |
| | PCR | Amplification of transcript using specific |
| | | primers. Can be degenerate mixtures. Requires |
| | | some prior knowledge. |

Protein

| | | |
|---|---|---|
| Overall display methods | 2-D gels | Separates on isoelectric point and size |
| | Capillary electrophoresis | Very sensitive size separation |
| | MS Ion trap | Extremely sensitive size separation |
| General means of protein | MS | |
| characterization | Tryptic digest | followed by MS and/or peptide sequencing |
| Specific proteins | Antibody detection | Need purified protein or close relative |
| | Enzyme activity | Enzyme assay |

In the events from application of a particular stimulus to a expression of a particular phenotype, there can be several interacting factors, as shown in Table 2, that are taken into account when determining how and when gene expression is to be measured.

TABLE 2

| Event | Factors | Measurement |
|---|---|---|
| Transcription of transcription | Time course; interaction with | Early post-stimulus assessment |
| factor-encoding genes | activators and repressors; half | of differential expression; |
| | life of mRNA | probing for mRNAs encoding |
| | | known classes of transcription |
| | | factors |
| Transcription of biosynthetic | Time course; interaction with | Later post-stimulus assessment |
| enzyme-encoding genes | activators and repressors; half | of differential expression; |
| | life of mRNA | probing for mRNAs encoding |
| | | known enzymes |
| Translation to enzyme protein | Time course; half-life of protein | Specific antibodies; proteomics |
| Enzyme activity | Cofactors; inhibitors; activators | Enzyme assay; end product |
| | | chemical assays |
| Production of chemical | Presence of entire pathway; time | Chemical assay |
| | course; half-life of chemical | |
| | (instability, catabolism) | |

For example, expression of genes encoding transcription factors by an external stimulus is generally an early response, while expression of genes encoding biosynthetic enzymes occurs later. Therefore, it is desirable to use a variety of external stimuli with inducing or non-inducing effects on the desired phenotype and to measure differential gene expression at different intervals after treatment with each stimulus. It is also desirable that the various target phenotype-inducing stimuli produce different patterns of gene expression. Induced transcripts that are common to all target phenotype-inducing stimuli and not to non-inducing stimuli are likely to be involved in the cellular response to the stimulus and, hence, the generation of the desired phenotype.

We have employed two general methods for identification of differentially expressed genes: “display methods” and “probing methods.” Display methods aim to show all, or a substantial proportion, of the total mRNAs (as cDNAs) in an expression profile. While this type of display provides no information about the nature of the genes, it does allow large numbers of mRNAs from control and variously treated cultures to be displayed alongside one another, rendering it possible to determine which mRNAs are expressed constitutively, and which are induced or repressed by any particular treatment.

Probing methods (e.g., PCR) are useful to demonstrate that the relevant pathway is activated by a treatment that results in a particular phenotype (i.e., that the induction occurs via altered gene expression). To employ probing methods, one needs sequence information for one or more genes in the pathway. The sequence may be from the species employed in the assay, from another related species, or from the consensus sequence from several related or unrelated species.

### EXAMPLE 1

### Differential Induction of EGCG in a Plant Cell Suspension Culture of Crassula fascicularis

A plant cell culture of Crassula fascicularis (Crassulaceae) was prepared using seeds of C. fascicularis. The seeds were sterilized by 15 minutes immersion in 5% Domestos (Lever Faberge, UK) with an active chlorine concentration of 0.25%. Sterile seeds were placed on seed germination media B83 (modified after Gamborg's B5 recipe+sucrose (1%), no hormones) containing propiconazole (10 mg/L). Three weeks later, following seed germination and limited root and shoot growth, the sterile seedlings were chopped into small pieces of approximately 5 mm and placed upon solidified callus induction medium B50 (modified after Gamborg's B5 recipe to contain 2,4-dichlorophenoxyacetic acid (2,4-D) (1 mg/L), kinetin (0.1 mg/L), coconut water (100 mL/L), and sucrose (2%)). Upon establishment of callus, the material was used to initiate suspension cultures.

To establish suspension cultures, portions of established callus were placed in 100 mL conical flasks containing liquid medium B105, modified after Gamborg's B5 recipe (Exp. Cell. Res. 50: 148, 1968) to contain 2,4-D (1 mg/L), kinetin (0.1 mg/L), coconut water (100 mL/L), glutamine (10 mM), and 3% sucrose. The liquid medium was replenished at 14 day intervals. After six weeks, the established suspension culture was routinely maintained in a 250 mL conical flask, by transferring 40 mL of 14 day old suspension culture into 100 mL fresh B105 medium. The culture was incubated at 25° C. in continuous dark and shaken at 140 rpm.

Differential induction of catechins in the C. fascicularis suspension culture was performed in 500 mL conical flasks containing 190 mL of either growth medium B105 (modified Gamborg's B5 medium containing 2,4-D (1 mg/L), kinetin (0.1 mg/L), coconut water (100 mL/L), glutamine (10 mM), and 3% sucrose) or a secondary metabolite production medium B49 (Gamborg's B5, 5% sucrose, no hormones), inoculated with 70 mL of 14 day old suspension culture. Cultures were grown for 14 days before harvest and processing. Further cultures grown on production medium B49 were treated using one of each of the following protocols:

(1) Seven days following inoculation, filter-sterilized methyl jasmonate (250 μM final concentration) was added;

(2) Seven days following inoculation, filter-sterilized methyl jasmonate (250 μM final concentration) and an autoclaved, non-viable, Candida albicans preparation (C. albicans) (50 mg/L final concentration) were added. The C. albicans was obtained by growing a culture of strain ATCC28516 on YEPD media (yeast extract 1%, yeast peptone 2%, glucose 2%) to maximal cell density and twice autoclaving the total yeast culture prior to addition to plant cultures; or

(3) Seven days following inoculation, filter-sterilized methyl jasmonate (250 μM final concentration) and filter-sterilized omithine (250 mg/L final concentration) was added.

An additional culture was treated using the following protocol prior to growth on production medium B49:

(4) A 40 mL aliquot of a day 0 suspension growing on B105 medium was transferred to a 100 mL flask. On day 3, a sterile solution of 5-azacytidine (5-AC) in water was added for a final concentration of 3×10⁻⁵ M, and the resultant mixture was incubated for 11 days. At this point the 40 mL 5-AC-treated culture was subcultured twice before inoculating 190 mL B49 production medium in a 500 mL flask with 70 mL of day 14 suspension. The culture was then treated further according to protocol (2).

The cell cultures were harvested by vacuum filtration after a further seven days of incubation.

Extraction and Sample Preparation

Lyophilized biomass from 100 mL plant cell culture was extracted three times with 10 mL of boiling aqueous 0.2 M NaH₂PO₄ solution (total 30 mL). The combined, cooled, extracts were extracted with EtOAc (2×30 mL). The combined EtOAc extracts were then dried under a stream of nitrogen. The samples were dissolved in 50% aqueous acetonitrile at a concentration of 1.0 mg/mL; a 0.1 mL aliquot was transferred into a 0.25 mL glass insert to a 2 mL HPLC vial. The extraction procedure is scalable according to the initial volume of the plant-cell culture used to generate the biomass.

HPLC Analysis

HPLC analyses of compounds in plant cell culture extracts were performed in two systems. System 1 (S1) utilized a Rainin Dynamax SD-200 pumping system, a Varian Dynamax PDA-2 diode array detector, with a Waters XTerra RP18 (5 μm, 3.0×150 mm) column. The mobile phase was composed from 0.1% acetic acid in H₂O (solvent A) and 0.1% acetic acid in acetonitrile (solvent B), at a flow rate of 0.75 mL/min: after maintaining initial conditions (A:B, 95:5) for one minute post-injection, gradient elution was accomplished in a linear fashion over 30 min (to A:B, 40:60). A total of 10 μL of each sample was injected onto the column and eluates were analyzed at a wavelength of 275 nm. System 2 (S2) utilized API-electrospray-LC-MS: a Hewlett Packard series HP1100 with a Waters Xterra RP18 (3.5 μm, 2.1×100 mm) column was used as the inlet for a Micromass Platform-LC, in the positive-ion mode. The gradient system utilized the same mobile phase at a flow rate of 0.25 mL/min and a sample injection of 5 μL. After two minutes flow at 90:10 (A:B), the percentage of B was increased linearly to 95% over 34 minutes. Compounds of interest were detected at 254 nm and as single-charged species via selected-ion monitoring in the mass spectrometer. See Table 4 for retention times, UV wavelengths, and m/z values.

Results

The catechin derivative, epigallocatechin (EGCG), was detected in samples treated with treatments (1), (3) and the control culture to which no additions were made. EGCG was not detected in samples treated with treatments (2) and (4), indicating that the addition of C. albicans likely reduced expression of the EGCG biosynthetic pathway.

Alternative Procedure

Differential induction of catechin derivatives in the C. fascicularis suspension culture was also performed in 250 mL conical flasks containing 90 mL of either growth medium B105 (modified Gamborg's B5 medium containing 2,4-D (1 mg/L), kinetin (0.1 mg/L), coconut water (100 mL/L), glutamine (10 mM), and 3% sucrose) or a secondary metabolite production medium B49 (Gamborg's B5, 5% sucrose, no hormones), inoculated with 40 mL of 14 day old suspension culture. After incubation under standard environmental conditions, on day 7 following subculture suspensions were treated using one of the protocols in Table 3.

TABLE 3

| Medium | | EGCG (mg/gdwt) two days |
|---|---|---|
| Type | Treatment Description | after treatment |
| B105 | growth medium (control) | 0 |
| B49 | production medium (control) | 80 |
| B49 | methyl jasmonate¹ | 67 |
| B49 | methyl jasmonate + C. albicans² | 30 |
| B49 | methyl jasmonate + ornithine³ | 97 |
| B49 | C. albicans | 32 |
| B49 | Clofibrate⁴ | 89 |
| B49 | FeSO₄.7H₂O⁵ | 90 |
| B49 | gallic acid⁶ | 82 |
| B49 | methyl jasmonate + 2,4-D⁷ | 56 |
| B49 | cold (16° C.)⁵ | 154 |
| B49 | continuous high light (160 lux)⁸ | 79 |

¹Jasmonates are signal molecules involved in the plant response to certain environmental stresses.
²C. albicans is an example of a biotic elicitor of the plant defense response.
³Some species of the family Crassulaceae are reported to accumulate piperidine alkaloids (e.g., sedamine) of which ornithine is a biosynthetic precursor.
⁴Clofibrate is an inducer of cytochrome p450 enzyme activity. Others include phenobarbital, manganese chloride, ethanol.
⁵FeSO₄.7H₂O and cold are known inducers of oxidative stress.
⁶Precursors of secondary metabolites have been shown to upregulate biosynthesis.
⁷2,4-D is reported to suppress secondary metabolism at the transcriptional level.
⁸High light induces greening and chloroplast differentiation in plants.

Subsequent re-growths were made from suspension cultures routinely maintained in 250 mL conical flasks by transferring 40 mL of 14 day old suspension culture into 100 mL fresh B105 medium, and incubating the culture at 25° C. in continuous dark and shaking at 140 rpm. Alternately, re-growths were made using material that had undergone several rounds of short-term cold storage, whereby 140 mL of a 3-day old culture was placed in a flat 600 mL tissue culture flask with vented lid and then stored at 15° C. for 91 days. The culture was then removed and placed in a 250 mL conical flask with media being replaced at 14 day intervals until the culture could be routinely maintained by transferring 40 mL of 14 day culture into 100 mL fresh B105 medium. At this point the cultures were either re-stored or re-scaled up as described earlier. In the latter case they were harvested, extracted and the extracts were analyzed as described in the initial procedure described above.

EGCG was detected in all treatments except the growth medium control. As described above for this culture, C. albicans-containing treatments reduced EGCG expression.

TABLE 4

| Compound | | | Retention time* | UV** | | |
|---|---|---|---|---|---|---|
| No. | Name | Structure | (min) | (nm) | m/z^{#} | Product Ions^{#} |
| 1 | Catechin | 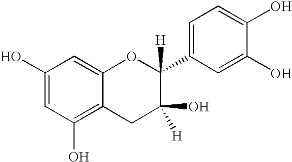 | S1 11.253 S2 7.95 | 275 | 291 | M + 1 |
| 2 | Epicatechin | 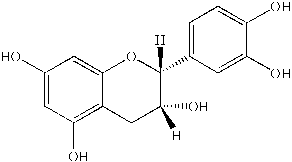 | S1 12.238 S2 13.20 | 275 | 291 | M + 1 |
| 3 | Epigallocatechin Gallate EGCG | 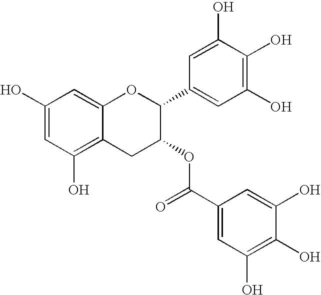 | S1 14.133 S2 14.30 | 274 | 459 289 | M + 1 M—C₇H₅O₅ |
| 4 | Gallocatechin GC | 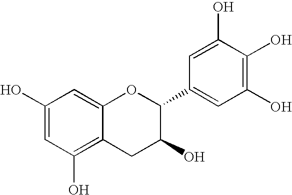 | S1 15.104 S2 15.31 | 274 | 307 | M + 1 |
| 5 | Epicatechin Gallate ECG | 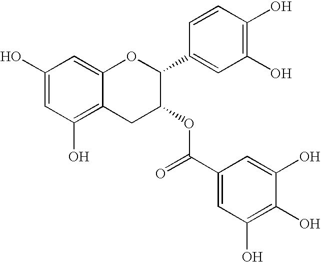 | S1 16.160 S2 17.02 | 274 | 443 273 | M + 1 M—C₇H₅O₅ |

*S1 and S2 refer to retention times in System S1 and System S2, respectively
**Compounds have UV maxima at the indicated wavelength.
^{#}Compounds were detected via LC/MS having these characteristics (m/z is the mass to charge ratio and M is the Molecular mass in Daltons) of the indicated compound.

Identification of additional culture conditions (i.e., external stimuli) that have the desired phenotype (e.g., increased production of any of Compounds 1-5) is performed using the methods described above. Once these phenotype-inducing conditions are identified, genes having altered expression under these conditions (and not under conditions that do not induce increased production of Compounds 1-5) can be identified using standard techniques, as are described herein.

### EXAMPLE 2

### Differential Induction of EGCG in a Plant Cell Suspension Culture of Crassula barkleyi

A plant cell culture of Crassula barkleyi (Crassulaceae) was prepared using shoots of C. barkleyi. The shoots were sterilized by immersion for one minute in 70% ethanol, then for 20 minutes in an Inov'chlor solution (Inov'Chem SA, Tanneries Cedex, France) with an active chlorine concentration of 1.05%. The sterile shoots were chopped into small pieces of approximately 5 mm and placed upon solidified callus induction medium B5 (Gamborg's B5 recipe containing 2,4-D (1 mg/L), kinetin (0.1 mg/L), sucrose (2%)). Callus initiations were incubated in the dark at 25° C. Upon establishment of callus, this was used to initiate suspension cultures.

To establish suspension cultures, portions of established callus were placed in 100 mL conical flasks containing liquid medium B105, modified after Gamborg's B5 recipe to contain 2,4-D (1 mg/L), kinetin (0.1 mg/L), coconut water (10%), glutamine (10 mM) and 3% sucrose. The liquid medium was replenished at 14 day intervals. After five months, the established suspension culture was routinely maintained in a 250 mL conical flask, by transferring between 12 mL and 40 mL of 14 day old suspension culture into 100 mL fresh B105 medium. The culture was incubated at 25° C. in continuous dark and shaken at 140 rpm.

Differential Induction of Accumulation of Catechins

Differential induction of catechin derivatives in the C. barkleyi suspension culture was performed in 250 mL conical flasks containing 100 mL of a secondary metabolite production medium B49 (Gamborg's B5, with 5% sucrose, and no hormones), inoculated with 16 mL of 14 day old suspension culture. The cultures were incubated under low light conditions (approximately 30 lux) at 25° C. for seven days after inoculation. Cultures were then treated using one of each of the following protocols (untreated cultures were also maintained as controls):

(1) Filter-sterilized methyl jasmonate (250 μM final concentration) was added;

(2) Autoclaved aqueous 2,4-D (1 mg/L final concentration) was added;

(3) Filter-sterilized methanolic methyl jasmonate (250 μM final concentration) and autoclaved aqueous 2,4-D (1 mg/L final concentration) were added;

(4) Filter-sterilized aqueous DL-phenylalanine (200 mg/L) was added;

(5) Filter-sterilized aqueous DL-phenylalanine (200 mg/L) and filter-sterilized methyl jasmonate (250 μM final concentration) were added;

(6) Filter-sterilized aqueous zeatin (10 μM) was added;

(7) Filter-sterilized aqueous 1-aminocyclopropane-1-carboxylic acid (10 mg/L final concentration) was added;

(8) Filter-sterilized 24-epibrassinolide (1 mg/L final concentration) was added;

(9) Incubation at a temperature of 16° C.; or

(10) High light regime (approximately 160 lux).

Ten milliliter culture samples were taken at regular intervals for 17 days following the day of subculture to B49 medium. All culture samples were centrifuged for five minutes at 4000 rpm, and the cell residue was freeze-dried. Extraction, sample preparation, and HPLC analysis were performed as described in Example 1.

Results

Several treatments induced catechin biosynthesis and EGCG accumulation, notably methyl jasmonate (alone and in combination with DL-phenylalanine or 2,4-D), zeatin, and 24-epibrassinolide (FIGS. 1 and 2). As a single treatment, 2,4-D acid suppressed catechin biosynthesis.

### EXAMPLE 3

### Differential Induction of EGCG in a Plant Cell Suspension Culture of Crassula dejecta

A plant cell culture of Crassula dejecta (Crassulaceae) was prepared using seed of C. dejecta. The seeds were sterilized by immersion for 30 minutes in 6% Domestos, followed by rinsing with four changes of sterile distilled water. The seeds were gently crushed and placed on solidified callus induction medium modified after Murashige & Skoog's recipe (Physiol. Plant. 15: 473-497, 1962) to contain 1 mg/L 2,4-D, 0.1 mg/L kinetin, 5 μM gibberellic acid, and 2% sucrose. Callus initiations were incubated in the dark at 25° C. for six months. Upon establishment of callus, the material was used to initiate suspension cultures.

To establish suspension cultures, portions of established callus were placed in 100 mL conical flasks containing liquid medium B105, modified after Gamborg's B5 recipe (Exp. Cell. Res. 50: 148, 1968) to contain 2,4-D (1 mg/L), kinetin (0.1 mg/L), coconut water (10%), glutamine (10 mM), and 3% sucrose. The liquid medium was replenished at 14 day intervals. After six months, the established suspension culture was routinely maintained in a 250 mL conical flask by transferring 40 mL of a 21 day old suspension culture into 100 mL of fresh B105 medium. The culture was incubated at 25° C. in continuous dark and shaken at 140 rpm.

Differential Induction of Catechin Accumulation

Differential induction of catechin derivatives in the C. dejecta suspension culture was performed in 250 mL conical flasks containing 100 mL of B105 growth medium or 100 mL of a secondary metabolite production medium B49 (Gamborg's B5, with 5% sucrose, and no hormones), inoculated with 40 mL of 21 day old suspension culture. The cultures were incubated under low light conditions (approximately 30 lux) at 25° C. Cultures were treated using one of each of the following protocols:

(1) B105 growth medium (no additions) used as is;

(2) Filter-sterilized methanolic methyl jasmonate (250 μM final concentration) was added at day 7 to a culture grown on B105 medium;

(3) Filter-sterilized aqueous zeatin (10 μM) was added at day 7 to a culture grown on B105 medium;

(4) B49 production medium (no additions) used as is;

(5) Filter-sterilized methyl jasmonate (250 μM final concentration) was added at day 7 to a culture grown on B49 medium; or

(6) Filter-sterilized clofibrate (0.5 mM final concentration) was added at day 7 to a culture grown on B49 medium.

Ten milliliters of culture samples were taken at regular intervals for 17 days following the day of subculture to B105 or B49 medium. All culture samples were centrifuged for five minutes at 4000 rpm, and the cell residue was freeze-dried. Extraction, sample preparation, and HPLC analysis were performed as described in Example 1.

Results

No catechins were detected in cultures grown on B105 in the absence of additions. Catechins, including EGCG, were accumulated in cultures grown on B49 without additions. Methyl jasmonate induced EGCG and other catechins in cultures grown on both B105 and B49 media (FIG. 3). On B49, a ten-fold accumulation of EGCG was observed. Zeatin also induced catechin, gallocatechin, and EGCG in cultures grown on B105. Clofibrate added to B49-grown cultures completely suppressed catechin accumulation.

### EXAMPLE 4

### Catechin Profiling in a Plant Cell Suspension Culture of Crassula acinaciformis

A plant cell culture of Crassula acinaciformis (Crassulaceae) was prepared using seed of C. acinaciformis. The seeds were sterilized by immersion for 30 minutes in a solution of Inov'chlor, then germinated on a germination medium B83 modified after Gamborg's B5 recipe to contain half-strength minerals and organic components, 1% sucrose, and 0.7% agar. Germinated seedlings were cut into 0.5 cm sections and placed on callus induction media M2, modified after Murashige and Skoog's recipe (Physiol. Plant. 15: 473-497, 1962) to contain 1 mg/L 2,4-D, 0.1 mg/L kinetin, and 2% sucrose. Callus initiations were incubated in the dark at 25° C. for seven months. Upon establishment of callus, the material was used to initiate suspension cultures.

To establish suspension cultures, portions of established callus were placed in 100 mL conical flasks containing liquid medium M62, modified after Murashige and Skoog's recipe (Physiol. Plant. 15: 473-497, 1962) to contain 1 mg/L 2,4-D, 0.1 mg/L kinetin, and 3% sucrose. The liquid medium was replenished at 14 day intervals. After six months, the established suspension culture was routinely maintained in a 250 mL conical flask by transferring 40 mL of a 21 day old suspension culture into 100 mL fresh M62 medium. The culture was incubated at 25° C. in continuous dark and shaken at 140 rpm.

Effect of Medium and Methyljasmonate Induction on Catechin Accumulation

Catechin derivative profiles in the C. acinaciformis suspension culture were obtained from incubations in 250 mL conical flasks containing either 100 mL of M62 growth medium or 100 mL of a secondary metabolite production medium M33 (Murashige and Skoog (1962); modified to contain 5% sucrose and no hormones) inoculated with 40 mL of 21 day old suspension culture. The cultures were incubated under low light conditions (approximately 30 lux) at 25° C. Cultures were either grown without additions or M33-grown cultures were treated at day 7 with filter-sterilized methyl jasmonate (250 μM final concentration). 10 mL culture samples were taken at regular intervals for 16 days following the day of subculture to M33 medium. All culture samples were centrifuged for five minutes at 4000 rpm, and the cell residue was freeze-dried. Extraction, sample preparation, and HPLC analysis were performed as described in Example 1.

Results

The only catechins to be detected were (+)-catechin and, in low amounts, epicatechin. The medium composition had no significant effect on catechin level. The addition of methyl jasmonate on day 7 resulted in an approximate doubling of catechin level by day 16 (FIG. 4).

### EXAMPLE 5

### Differential Induction of RNA Transcripts in Plant Cell Suspension Cultures of Crassula Species

Differential induction of mRNA transcripts associated with the synthesis of catechin derivatives in the C. barkleyi suspension culture (as described in Example 2) and the C. dejecta suspension culture (as described in Example 3) were performed in 250 mL conical flasks containing 100 mL of a secondary metabolite production medium B49 (Gamborg's B5, 5% sucrose, no hormones), inoculated with 16 mL of a 14 day old suspension culture (C. barkleyi) or 40 mL of a 21 day old suspension culture (C. dejecta). The cultures were incubated under low light conditions (approximately 30 lux) at 25° C. Two days after inoculation, cultures were treated by the addition of methyl jasmonate (250 μM, final concentration), with untreated cultures also maintained as controls.

Induction of mRNA usually occurs within 48 hours of receipt of an inducing signal. Accordingly, samples were taken at intervals of 2, 4, 6, 10, 15, 24, and 48 hours following the above treatments. Samples, sufficient to give at least 1 mL packed cell volume, were centrifuged for five minutes at 4000 rpm and the supernatant removed. The residue was resuspended in five volumes of RNAlater™ (Ambion, Austin, Tex.) and frozen at −20° C. RNA remains stable indefinitely when treated in this fashion. RNA extraction was carried out with a Qiagen RNeasy® Plant Mini kit, using 4 columns per gram fresh weight of cells.

In order to verify the induction of genes involved in the biosynthesis of the intermediates en route to the biosynthesis of EGCG, we selected three proteins previously characterized in the literature. The first is L-phenylalanine ammonia lyase (PAL), which is the first step in the biosynthesis of phenylpropanoids; it deaminates phenylalanine to cinnamic acid. This enzyme has been cloned from numerous plants and has been shown to be highly inducible by various stress treatments. The second enzyme, chalcone synthase (CHS), is responsible for the condensation of three molecules of malonyl-CoA with one molecule of coumaroyl-CoA to yield chalcone. The third enzyme we chose is the flavanone-3 β-hydroxylase (F3-OH), a 2-oxoglutarate dependent dioxygenase that catalyses the 3β-hydroxylation of 2S-flavanones to 2R,3R dihydroflavonols. For each of these three enzymes, a set of plant-derived sequences have been retrieved from GenBank in order to find conserved nucleotide sequence regions among them. This led to the design of three pairs of oligonucleotides (one pair for each enzyme) used for RT-PCR amplification of messenger RNA corresponding to the three genes. The amplified PCR products were then sequenced and compared to the original alignments to verify gene identity. This allowed cloning of novel fragments of PAL, CHS and F3-OH from C. barkleyi and C. dejecta. (FIG. 5) Additionally, a fourth gene fragment corresponding to a gene whose expression is usually considered as invariant has been amplified using the same approach. This gene codes for the TATA binding protein factor (TBP).

RNA was extracted from cells harvested at different times after inducing treatments, together with untreated controls, and submitted to first strand synthesis using the following conditions:

1 μg total RNA

300 pmol of dT₁₈

QSP 25 μL DEPC-treated water

This was incubated at 70° C. for 10 min then Mix 1 was added.

Mix 1:

8 μL 1^{st}-strand buffer (Gibco BRL)

4 μL DTT (supplied with the kit)

2 μL dNTPs at 10 mM

1 μL Superscript™ II RT enzyme (Gibco BRL)

This mixture was incubated at 42° C. for 50 min then denatured at 70° C. for 15 min and stored at −20° C., after a thirty-fold dilution with distilled water. An aliquot of this reaction was then used to quantify the relative abundance of each of the three genes (PAL, CHS, F3-OH) to the invariant one (TBP), using a real time quantification PCR machine (Perkin Elmer 7700) and the previously described oligonucleotides (shown in FIG. 7) in the following conditions:

12.5 μL SYBR Green master mix (Perkin Elmer)

3 μL first strand reaction

40 pmol of primer A

40 pmol of primer B

QSP 25 μL distilled water.

(Primers A and B are the forward and reverse primers specific for the particular genes). This mixture is then cycled in the Perkin Elmer 7700 machine in the following conditions:

50° C. for 2 min

95° C. for 10 min

40 cycles of 95° C. for 15 sec

63° C. for 1 min

The results of the time course experiment for C. barkleyi are given in FIGS. 6A-6C. They show that cells of C. barkleyi respond to the addition of methyl jasmonate by inducing the transcription of PAL, CHS, and F3-OH at least twenty fold after 24 hours of treatment. Similarly, in C. dejecta all three genes are induced at least 6-fold by methyl jasmonate after 48 hours.

These experiments not only verify that genes on the catechin biosynthetic pathway are inducible by methyl jasmonate but also indicate that 48 hours post-induction would be a suitable time for looking for novel transcripts associated with EGCG production.

### EXAMPLE 6

### Detection of Catechins in Crassula barkleyi

An air-dried sample of whole plants of Crassula barkleyi (2.3 g) was extracted twice with hot methanol (100 mL portions). The combined extracts were concentrated to dryness on a rotary evaporator and the residue was digested in a mixture of distilled water (30 mL) and ethyl acetate (30 mL). The biphasic system was separated and the aqueous layer was extracted twice further with ethyl acetate (30 mL portions). The combined ethyl acetate extracts were evaporated to a residue (52 mg). HPLC/UV/MS analysis of the residue for catechin content was carried out as described in Example 1 for extracts of plant cell cultures.

Results

The following catechins were found to be present in the ethyl acetate extract, and were characterized by HPLC retention time, UV absorption spectrum and mass spectral detection of the parent and known degradation ions.

TABLE 5

| Catechin | % of Ethyl acetate extract |
|---|---|
| Gallocatechin | 3.1 |
| Epigallocatechin | 0.84 |
| Catechin | 0.13 |
| Epigallocatechin gallate | 1.2 |

### EXAMPLE 7

### EGCG Production and Catechin Profiling in a Plant Cell Suspension Culture of Sempervivum tectorum

EGCG has been reported as a possible component of the polymeric polyphenols isolated from leaves of the plant Sempervivum tectorum (Crassulaceae) (Abram et al., J. Agric. Food Chem. 47: 485-489, 1999).

Initiation of Suspension Plant Cell Cultures

A plant cell culture of S. tectorum was prepared using seeds of S. tectorum. The seeds were sterilized by immersion for 30 minutes in a 5% aqueous solution of Domestos, then germinated on a germination medium B83 modified after Gamborg's B5 recipe to contain half-strength minerals and organic components, 1% sucrose, and 0.7% agar. Germinated seedlings were cut into 0.5 cm sections and placed on callus induction media B50 modified after Gamborg's B5 recipe to contain 1 mg/L 2,4-D, 0.1 mg/L kinetin, 10% coconut water, and 2% sucrose. Callus initiations were incubated in the dark at 25° C. for five months. Upon establishment of callus, the material was used to initiate suspension cultures.

To establish suspension cultures, portions of established callus were placed in 100 mL conical flasks containing liquid medium B88, modified after Gamborg's B5 recipe to contain 1 mg/L 2,4-D, 0.1 mg/L kinetin, 10% coconut water, and 3% sucrose. The liquid medium was replenished at 14 day intervals. After four months, the established suspension culture was routinely maintained in a 250 mL conical flask by transferring 40 mL of a 21 day old suspension culture into 100 mL fresh B88 medium. The culture was incubated at 25° C. in continuous dark and shaken at 140 rpm.

Catechin derivative profiles in the S. tectorum suspension culture were obtained from incubations in 250 mL conical flasks containing either 100 mL of B88 growth medium or 100 mL of a secondary metabolite production medium B49 (Gamborg's B5 modified to contain 5% sucrose and no hormones) inoculated with 40 mL of 21 day old suspension culture. The cultures were incubated under low light conditions (approximately 30 lux) at 25° C. Cultures were either grown without additions or B49-grown cultures were treated at day 7 with filter-sterilized methanolic methyl jasmonate (250 μM final concentration).

Ten milliliters of culture samples were taken at regular intervals for 16 days following the day of subculture to B88 or B49 medium. All culture samples were centrifuged for five minutes at 4000 rpm and the supernatant liquid was removed and frozen. The cell residue was freeze-dried. Extraction, sample preparation, and HPLC analysis were performed as described in Example 1.

Results

Gallocatechin, epigallocatechin and EGCG were detected under all conditions, although only trace amounts were found in cultures grown on B88 medium. Cultivation on B49 production medium induced accumulation of all three compounds. The addition of methyl jasmonate on day 7 brought about no further increase in catechin level (FIG. 8).

### EXAMPLE 8

### Catechin Profiling in Plant Cell Suspension Cultures of Species from the Family Polygonaceae

EGCG has been reported in members of the plant family Polygonaceae (e.g., in Coccoloba dugandiana (Li et al., Planta Medica 65: 780, 1999), Polygonum multiflorum (Horikawa et al., Mutagenesis 9: 523-526, 1994), and rhubarb (Kashiwada et al., Chem. Pharmaceut. Bull. (Tokyo) 34: 4083-4091, 1986). Epicatechin and epicatechin gallate, but not EGCG, have been previously reported in callus or suspension cultures derived from members of the family Polygonaceae, specifically Fagopyrum esculentum (Moumou et al. Planta Medica 58: 516-519, 1992) and Polygonum hydropiper (Nakao et al. Plant Cell Rep. 18: 759-763, 1999).

Initiation of Suspension Plant Cell Cultures of Three Members of the Family Polygonaceae

A cell suspension culture of Fallopia convolvulus was prepared using shoot material of F. convolvulus. The shoots were sterilized by a pre-treatment in 96% ethanol for 30 seconds, followed by 15 minutes immersion in a 1% solution of Dimanin C (Bayer, Germany). Sterilized shoots were cut into 0.5 cm sections and placed on callus induction media B50 modified after Gamborg's B5 recipe to contain 1 mg/L 2,4-D, 0.1 mg/L kinetin, 10% coconut water, and 2% sucrose, also containing propiconazole (40 mg/L). Callus initiations were incubated in the dark at 25° C. until callus was established.

To establish suspension cultures, portions of established callus were placed in 160 mL conical flasks containing liquid medium B105, modified after Gamborg's B5 recipe to contain 1 mg/L 2,4-D, 0.1 mg/L kinetin, 10% coconut water, 10 mM glutamine and 3% sucrose. The liquid medium was replenished at 14 day intervals. After two months, the established suspension culture was routinely maintained in a 250 mL conical flask by transferring between 20 mL and 40 mL of a 14 day old suspension culture into 100 mL fresh B105 medium. The culture was incubated at 25° C. in continuous dark and shaken at 140 rpm.

A cell suspension culture of Rumex sagittatus was prepared using seeds of R. sagittatus. The seeds were sterilized by immersion for 30 minutes in a solution of Inov'chlor. The seeds were germinated on a germination medium B83 modified after Gamborg's B5 recipe to contain half-strength minerals and organic components, 1% sucrose, and 0.7% agar. Germinated seedlings were cut into 0.5 cm sections and placed on callus induction media B50 modified after Gamborg's B5 recipe to contain 1 mg/L 2,4-D, 0.1 mg/L kinetin, 10% coconut water, and 2% sucrose. Callus initiations were incubated in the dark at 25° C. until callus was established.

To establish suspension cultures, portions of established callus were placed in 100 mL conical flasks containing liquid medium B114, modified after Gamborg's B5 recipe to contain 1 mg/L 2,4-D, 0.1 mg/L kinetin, 0.55 mg/L thidaizuron, 10% coconut water, and 2% sucrose. The liquid medium was replenished at 14 day intervals. After eight months, the established suspension culture was routinely maintained in a 250 mL conical flask by transferring between 40 mL of a 21 day old suspension culture into 100 mL fresh B114 medium. The culture was incubated at 25° C. in continuous dark and shaken at 140 rpm.

A cell suspension culture of Rumex obtusifolius was prepared using seeds of R. obtusifolius. The seeds were sterilized by 30 minutes immersion in a 10.5% solution of Inov'Chlor, then germinated on water agar containing 1% coconut water and 0.7% agar. Germinated seedlings were then cut into 0.5 cm portions and placed on a callus induction media B58 modified after Gamborg's B5 recipe to contain 0.1 mg/L picloram and 2% sucrose. Callus initiations were incubated in the dark for three months at 25° C. until callus was established.

To establish suspension cultures, portions of established callus were placed in 100 mL conical flasks containing liquid medium B122, modified after Gamborg's B5 recipe to contain 1 mg/L 2,4-D, 0.1 mg/L kinetin, 10% cold water extract of banana powder (Sigma), and 3% sucrose. The liquid medium was replenished at 14 day intervals. After two months, the established suspension culture was routinely maintained in a 250 mL conical flask by transferring between 40 mL of a 14 day old suspension culture into 100 mL fresh B122 medium. The culture was incubated at 25° C. in continuous dark and shaken at 140 rpm.

Accumulation of Catechins

Catechin profiles in suspension cultures of R. sagittatus and R. obtusifolius were obtained from incubations in 250 mL conical flasks containing either 100 mL of their respective growth medium or 100 mL of a secondary metabolite production medium B49 (Gamborg's B5 modified to contain 5% sucrose and no hormones) inoculated with 40 mL of suspension culture grown for a complete growth cycle. The cultures were incubated under low light conditions (approximately 30 lux) at 25° C. Cultures were either grown without additions or cultures grown on B49 production medium were treated at day 7 with filter-sterilized methanolic clofibrate (0.5 mM final concentration). Ten milliliter culture samples were taken at regular intervals for 17 days following the inoculation day. All culture samples were centrifuged for five minutes at 4000 rpm and the cell residue was freeze-dried. Extraction, sample preparation, and HPLC analysis were performed as described in Example 1.

Results

Catechin, epicatechin, and epicatechin gallate were detected under all conditions (FIG. 9). Cultivation on B49 production medium induced accumulation of all three compounds.

### EXAMPLE 9

### Differential Induction of Diterpenes in a Plant Cell Suspension Culture of Ajuga reptans

A plant cell culture of Ajuga reptans (Labiatae) was prepared using young shoots of A. reptans. The shoot surfaces were sterilized by brief immersion in 70% ethanol followed by immersion in 15% sodium hypochlorite for 20 minutes. The sterilized shoots were chopped into small pieces approximately 5 mm long and placed upon solidified callus induction medium B39, modified after Gamborg's B5 recipe to contain 2,4-dichlorophenoxyacetic acid (2,4-D) (5 mg/L), sucrose (2%), and 0.5% gelrite (Duchefa Biochemie BV, Haarlem, the Netherlands). Upon establishment of callus, the material was used to initiate suspension cultures.

To establish suspension cultures, portions of established callus were placed in 100 mL conical flasks containing 20 mL liquid medium B39. The liquid medium was replenished at 7-14 day intervals. After two months, the established suspension culture was routinely maintained in a 250 mL conical flask, by transferring between 16 and 40 mL of 14 day old suspension culture into 100 mL fresh B39 medium. The culture was incubated at 25° C. in continuous low light and shaken at 140 rpm.

Differential induction of diterpenoids in the Ajuga reptans suspension culture was performed by inoculating 500 mL conical flasks containing 190 mL of a secondary metabolite production medium B49 (Gamborg's B5, 5% sucrose, no hormones), with 70 mL of 14 day old suspension culture. The culture was incubated at 25° C. in continuous low light and shaken at 140 rpm. Cultures were grown for seven days (25° C. in continuous low light and shaken at 140 rpm) and then dispensed as 5 mL aliquots to 6-well plates (Bibby Sterilin Ltd, Stone, UK). Cultures were then treated using one of the following protocols:

(1) No additions;

(2) Seven days following inoculation, filter-sterilized methyl jasmonate (250 μM final concentration) was added;

(3) Seven days following inoculation, C. albicans preparation (50 mg/L final concentration) were added; or

(4) Seven days following inoculation, filter-sterilized methyl jasmonate (250 μM final concentration), and an autoclaved C. albicans preparation (50 mg/L final concentration) were added.

Triplicate samples were set up for each treatment. Cultures were incubated for a further seven days before harvest for extraction and analysis of diterpenoids.

Differential induction of diterpenoids in the A. reptans suspension culture was also performed using one of the protocols in Table 6.

TABLE 6

| Treatment Description | Class of Treatment |
|---|---|
| Polyethylene glycol | inducer of desiccation, osmotic stress |
| salicylic acid | stress signaling molecule in plants |
| gibberellic acid | plant hormone inducing growth and |
| | differentiation |
| 2,4-D | synthetic phytohormone suppressing secondary |
| | metabolism |
| cellulase | example of a biotic elicitor inducing pathogen |
| | defense response |
| clofibrate | inducer of cytochrome p450 enzymes |
| Cerium (IV) oxide | Reported inducer of secondary metabolism |
| FeSO₄.7H₂O | inducer of oxidative stress |
| aphidicolin | cell cycle inhibitor |
| zeatin | plant hormone inducing differentiation |
| aminocyclopropane | precursor of ethylene (stress inducer) in plants |
| carboxylic acid | |
| cold (16° C.) | inducer of oxidative stress |
| continuous high | inducer of plastid differentiation |
| light (160 lux) | |

Subsequent re-growths were made from suspension cultures routinely maintained in 250 mL conical flasks by transferring 40 mL of 14 day old suspension culture into 100 mL fresh B39 medium, and incubating the culture at 25° C. in continuous low light and shaking at 140 rpm.

Cultures were harvested by freezing entire 6-well plates at −20° C. and then freeze-drying cultures in situ. Diterpenoids and other low molecular weight constituents were extracted by adding 5 mL Analar methanol and incubating overnight at room temperature. Following evaporation to dryness, a further 5 mL Analar methanol was added. After standing for one hour 500 μL of filtered methanol extract was removed and immediately analyzed.

HPLC analysis was carried out using an Xterra RP18 column (dimensions 3×150 mm with 5 μm packing) using an isocratic solvent system of water (two volumes):acetonitrile (three volumes) to which has been added 0.1% acetic acid. A flow rate of 0.75 mL/min was maintained throughout a ten minute chromatogram. The eluate was monitored by UV absorption. Identified compounds are shown in Table 7. The principal diterpene metabolite (Compound 6) has UV absorption maxima at 230, 290 and 340 nm and can be sensitively monitored at any of these wavelengths. Compound 6 has a retention time of 7.7 minutes in this system. Most of the minor diterpenes elute after Compound 6, with the exception of compound 7, which elutes earlier. Another HPLC system that can be employed uses a Waters reversed phase u-Bondapak C-18 column (dimensions 3.9×300 mm with isocratic acetonitrile:water 3:2 at a flow rate of 1 mL/min). Under these conditions, Compound 6 elutes at approximately 20.5 minutes and compound 7 at approximately 16 minutes.

TABLE 7

| | | |
|---|---|---|
| Compound 6 | 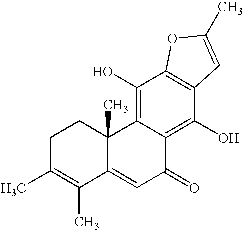 | Major component |
| Compound 7 | 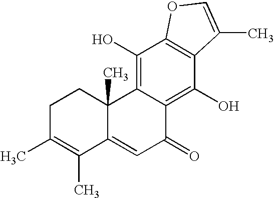 | Significant minor |
| Compound 8 | 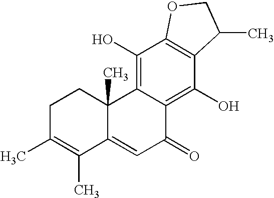 | Minor component |
| Compound 9 | 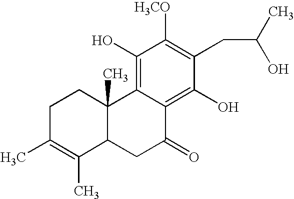 | Minor component |
| Compound 10 | 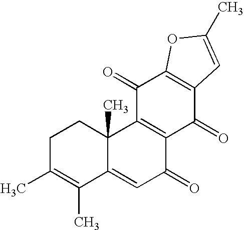 | Minor component |
| Compound 11 | 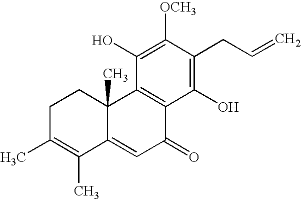 | Minor component |
| Compound 12 | 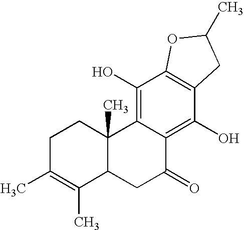 | Minor component |

The amount of compound 6 produced under conditions is summarized in Table 8, below.

TABLE 8

| | Compound 6 (mg/L culture) | |
|---|---|---|
| Treatment (n = 3) | Mean | (SD) |
| Production medium only | 0 | |
| +methyl jasmonate | 9.52 | (3.08) |
| +C. albicans | 166.12 | (20.70) |
| +methyl jasmonate + C. albicans | 137.01 | (3.6) |

Identification of additional culture conditions (i.e., external stimuli) that have the desired phenotype (i.e., increased production of Compound 6) is performed using the methods described above. Once these phenotype-inducing conditions are identified, genes having altered expression under these conditions (and not under conditions that do not induce increased production of Compound 6) can be identified using standard techniques, as is described herein. Using these same methods, one can identify culture conditions that increase the production of any of Compounds 7-12, as well as culture conditions that do not increase their production.

### EXAMPLE 10

### Differential Gene Expression of Transcripts in Ajuga reptans

Production of mRNA transcripts following a particular treatment generally occurs within 48 hours of the application of the particular treatment. Therefore to assess changes in the mRNA profile total RNA was prepared from cell cultures of Ajuga reptans treated as above in 6-well plates and harvested 24 h after treatment.

The contents of each well were harvested on to filter paper held in stainless steel filtration units and immediately frozen in liquid nitrogen. Total RNA was prepared using an RNeasy™ kit (Qiagen, Valencia, Calif.) as follows. The frozen biomass was first ground to a fine powder whilst still frozen in a porcelain pestle and mortar chilled in liquid nitrogen. Approximately 100 mg of the powder was used to prepare RNA. The yield was calculated by the absorbance at 260 nm (A₂₆₀=1 corresponds to 40 μg mL⁻¹). Yields were typically 10-30 μg per 100 mg biomass. mRNA profiles were displayed using two different display methods, AFLP and differential display, the results of which are described below.

AFLP

The amplified fragment-length polymorphism (AFLP) technique (Vos et al., Nucleic Acids Research 23: 4407-4414, 1995) was conducted as follows. The first strand cDNA was made using a biotinylated polyT primer, and the cDNA cut using BstYI, an enzyme that cuts approximately every 1 kb. The biotinylated 3′ ends of the cDNAs were selected using streptavidin, ensuring that only a single band is produced per transcript, and the resulting fragments further cut with MseI, an enzyme that cuts on average every 0.25 kb. It is calculated that 50-60% of all transcripts will produce fragments. The coverage can be increased using different enzyme combinations. The fragments were then ligated to specific adapters and amplified by PCR. If the average cell contains 10,000 different transcripts then about 5,000 different transcripts would be selected by this method. If desired, the specificity of the priming can be increased through the use of one or more anchoring bases. In the present example, two bases were used at one end (16 different combinations) and one at the other (four different combinations), thus generating 64 different primer combinations. Using this approach, the number of different transcripts per primer combination is reduced to approximately 80, which corresponds to the number of transcripts that can be detected on a single gel. Theoretically, a transcript that is present in only a single copy per cell can be detected. An example is shown in FIG. 10.

Transcripts differentially amplified following different treatments were selected by eye. About 1.2% (˜60 per display) of all bands showed very clear differences, while about 4.4% (˜220 per display) were scored as differences in the next selective cut. Some clear regulatory patterns emerged. Approximately half of all differentially expressed bands were up-regulated by one or more treatments, and approximately half were down-regulated, compared with the untreated control (Table 9). Most of the bands up-regulated following treatment with C. albicans were also upregulated following treatment with methyl jasmonate and C. albicans; most of these were not up-regulated by methyl jasmonate alone, so that 25% of all differentially regulated bands were correlated with the production of compound 6. In contrast, a substantial number of transcripts (˜15%) were up-regulated following treatment with methyl jasmonate alone but not by methyl jasmonate and C. albicans. The 10% of transcripts upregulated by all three treatments can be eliminated from further consideration, although they would have correlated with the production of Compound 6 on the basis of the C. albicans results alone (Table 9).

TABLE 9

| | % of total | | | | | | |
|---|---|---|---|---|---|---|---|
| | differentially | | | | | | |
| Main subsets of | expressed | | | | | | |
| differentially | transcripts | | Culture | | | | Up-regulated |
| expressed | represented by | | conditions* | | | | transcripts correlate |
| transcripts | each subset | | 1 | 2 | 3 | 4 | with phenotype? |
| A | | 25% | | | ✓ | ✓ | YES |
| B | Approx | 25% | ✓ | ✓ | | | No |
| C | Approx | 25% | ✓ | | | | No |
| D | | 15% | | ✓ | | | No |
| E | Approx | 10% | | ✓ | ✓ | ✓ | No |
| | TOTAL | 100% | | | | | |

*1 = Production media
2 = Methyl jasmonate
3 = C. albicans
4 = Methyl jasmonate + C. albicans

Differential Display

Differential display analysis was carried out as described below. Each of the four treatments described above were amplified with 122 primer combinations and analyzed on 15 gels. The primer combinations consisted of 24 specific ‘upstream’ primers combined with a non-specific downstream primer consisting of 11 polyT (to recognize the 3′ polyA tail on the mRNA) and one or two anchoring bases. The bands counted were around 80 per track, giving an overall number of around 10,000, which approximated the expected 10,000-15,000 transcripts. The size range detected by the gels was in the range 150-1000 bases.

In the eight gels scored, about 112 differentially expressed bands were found, corresponing to about 3% of the total bands. Thirty-seven percent of all differences were positively correlated with chemistry (i.e., upregulation in treatment with C. albicans, and with C. albicans+methyl jasmonate). Another portion (23%) correlated with the addition of methyl jasmonate alone. These results agree well with those obtained from AFLP.

A selection of differentially expressed bands was extracted and sequenced, and the sequences compared to those in the public gene databases using the alignment program FASTA. Forty-five sequences were obtained, 39 of them unique. Ten matching alignments were obtained, of which seven compared with known plant cDNA sequences (Table 10). The 29 non-aligned sequences could be novel genes, but most likely do not match because they are non-coding regions. Because Differential Display uses an oligo-dT primer all the bands will include the non-coding 3′ end of the mRNA.

TABLE 10

| | Transcript | | | | |
|---|---|---|---|---|---|
| | found in culture | | | | |
| | conditions | | | | |
| | (as defined | | | | Up-regulated transcripts |
| Putative identity based on | in Table 9): | | | | correlate with |
| alignment | 1 | 2 | 3 | 4 | phenotype? |
| alternate oxidase | | | | ✓ | No |
| β-glucosidase | ✓ | | | | No |
| receptor-like kinase | | ✓ | | | No |
| ubiquitin-activating protein | ✓ | | | | No |
| SCARECROW protein | | ✓ | | | No |
| Cu (cation) transporting | | | ✓ | ✓ | Yes |
| ATPase | | | | | |
| Membrane channel protein | | ✓ | | | No |
| of MIP family | | | | | |

RACE

Some of the differentially-regulated bands were subject to the RACE (rapid amplification of cDNA ends) procedure using a commercial RACE kit (Boehringer-Mannheim) in an attempt to obtain full-length cDNAs. RACE fragments were obtained from five previously-unidentified, differentially-regulated bands, and these were subsequently matched to sequences in the EMBL sequence banks. The putative identifications are shown in Table 11. One of the differentially-regulated genes was identified as a terpene cyclase, probably a monoterpene cyclase, but, because the regulation pattern did not correlate with the production of compound 6, it does not appear that this cyclase is specific for that product.

TABLE 11

| Regulation | | | | | |
|---|---|---|---|---|---|
| | C. | Methyl | C. albicans + | Putative | |
| control | albicans | jasmonate | methyl jasmonate | Identity | Comments |
| − | − | + | − | Lectin | |
| + | − | + | − | MAD2 | homolog of yeast cell cycle checkpoint |
| | | | | | protein |
| − | + | − | + | trypsin | |
| | | | | inhibitor | |
| + | − | − | − | cyt P450 | |
| − | − | + | − | terpene | best match = monoterpene cyclase, but also |
| | | | | cyclase | has homology to sesquiterpene and |
| | | | | | diterpene cyclases. |

Polymerase Chain Reaction

To investigate the pattern of regulation of the terpene cyclase identified as described above, primers were synthesized that corresponded to sequence at each end of the RACE fragment. Ajuga reptans cultures were subject to the treatments described above, and RNA collected eight and 24 hours after treatment. RT-PCR was carried out using an ADVANTAGE™ One-Step RT-PCR kit (Clontech Laboratories, Palo Alto, Calif.). The results show that the terpene cyclase is induced by methyl jasmonate and by methyl jasmonate+C. albicans 24 hours after treatment (FIGS. 11A and 11B), thus confirming the differential display results. The primers also appear to be amplifying a constitutive cyclase.

### EXAMPLE 11

### Use of Degenerate Primers to Find Terpene Cyclases

A large number of terpene cyclases have been sequenced and the sequences found to fall into three distinct classes (Trapp et al., Genetics 158: 811-832, 2001). Most angiosperm mono-, sesqui- and di-terpene cyclases fall within Class III. Consequently, degenerate primers were chosen from conserved sequences from four angiosperm sequences: limonene (monoterpene) synthase from Mentha spicata (GenBank L13456), vetispiradiene (sesquiterpene) synthase from Solanum tuberosum (GenBank AF042382), casbene (diterpene) synthase from Ricinus communis (GenBank L32134), and the RACE clone from Ajuga reptans. RNA samples (2 μg) from 24 hour post-treatment cultures of A. reptans were reverse transcribed using an Omniscript™ RT kit (Qiagen, Valencia, Calif.), with an oligo-dT₁₂₋₁₈ primer (Sigma-Aldrich Company, Poole, Dorset, UK). Aliquots (≡0.2 μg RNA) were subject to PCR using a Taq PCR Master Mix kit (Qiagen) and combinations of forward and reverse degenerate primers. Two primer combinations results in PCR bands: one induced by methyl jasmonate and not by C. albicans cell wall, and the other induced by C. albicans and not by methyl jasmonate (FIG. 12). The methyl jasmonate-induced band is very likely to be the same as the RACE clone described above, while the C. albicans-induced band is a strong candidate for the specific cyclase associated with compound 6 production.

### EXAMPLE 12

### Differential Induction of a Taxadiene Cyclase Homologue in a Plant Cell Suspension Culture of Taxus baccata

A plant cell culture of Taxus baccata was prepared using shoot material of Taxus baccata L. var “Rushmore.” The shoots were surface-sterilized by immersion for 20 minutes in a solution of 15% Domestos (Unilever, Lever Fagergé, UK) then washed thoroughly with sterile distilled water. Sterile shoots were chopped into small pieces of approximately 5 mm and placed upon solidified callus induction medium B12 modified after Gamborg's B5 recipe to contain 2,4-D (1 mg/L), kinetin (0.1 mg/L), coconut water (100 mL/L), sucrose (2%) and agar (1%). Upon establishment of callus, the material was used to initiate suspension cultures. To establish suspension cultures, portions of established callus were placed in 100 mL conical flasks containing liquid medium B50, modified after Gamborg's B5 recipe to contain 2,4-D (1 mg/L), kinetin (0.1 mg/L), coconut water (100 mL/L), and 2% sucrose. The liquid medium was refreshed at 14 to 21 day intervals for five months, at which point the established suspension culture was routinely maintained in a 250 mL conical flask, by transferring 40 mL of 14 day old suspension culture into 100 mL of fresh B50 medium at 21 day intervals. The culture was incubated at 25° C. in continuous low light and shaken at 140 rpm.

Homogeneous callus cultures were established from suspension cultures by transferring 4 mL of suspension culture onto the surface of B50 medium solidified with agar. The cultures were incubated at 25° C. in continuous low light on the shelf, and maintained by subculturing 1 cm³ portions of callus to fresh solid B50 medium at four week intervals.

Thirteen-day-old callus cultures prepared as described above were treated with methyl jasmonate by the addition of 10 μL of a 1:10 dilution of methyl jasmonate to a filter placed on the agar surface. Callus was harvested 24 and 48 hours after treatment. An untreated control was also harvested. Samples (500 mg) were frozen in liquid nitrogen and ground to a fine powder. RNA was prepared with an RNEASY™ kit (Qiagen, Valencia, Calif.) as described above.

RNA samples (1.75 μg) from 24 hours post-treatment cultures of Taxus baccata were reverse transcribed using an OMNISCRIPT™ RT kit (Qiagen) with an oligo-dT₁₂₋₁₈ primer (Sigma-Aldrich Company, Poole, Dorset, UK). Aliquots (≡0.175 μg RNA) were subject to PCR using a Taq PCR Master Mix kit (Qiagen). Primers were designed from conserved regions of the taxadiene synthases from Taxus chinensis and Taxus brevifolia, and validated by amplification of DNA from Taxus baccata. PCR was carried out at an annealing temperature of 50° C. for 30′; extension was for 1 min at 72° C. Samples were taken after 15, 20, 25, and 30 cycles and run on an agarose gel (FIG. 13). After 20 cycles, a band of the expected size is present in the treated samples (slightly more in the 24 hour harvest) but not in the control. After 25 cycles, a band in the control lane is present, but the bands in the treated samples are clearly more intense, showing a specific induction of the taxadiene cyclase by the methyl jasmonate treatment. These results are consistent with previous reports of methyl jasmonate-induced production of taxanes in cultures of a number of Taxus species (Yukimune et al., Phytochemistry 54:13-17, 2000; Walker et al., PNAS 97:583-587, 2000).

### Other Embodiments

All publications mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in plant cell culture or related fields are intended to be within the scope of the invention.

What is claimed is:

1. A method for identifying a gene associated with a desired phenotype, said method comprising the steps of:
(a) providing a plurality of cell cultures, each comprising plant, animal, or fungal cells capable of exhibiting said desired phenotype;
(b) contacting cells of step (a) with a stimulus that either (i) induces said cells to exhibit said phenotype, or (ii) does not induce said cells to exhibit said phenotype;
(c) determining the presence of the desired phenotype in the cells of step (b); and
(d) identifying a gene in said cells that has increased expression in response to stimuli that induce said phenotype but does not have increased expression in response to stimuli that do not induce said phenotype, wherein said identified gene is associated with said desired phenotype.

2. The method of claim 1, wherein said cells are plant cells.

3. The method of claim 2, wherein said phenotype is the production of terpenes.

4. The method of claim 3, wherein said terpenes comprise monoterpenes, diterpenes, or sesquiterpenes.

5. The method of claim 2, wherein said plant cells comprise Ajuga reptans cells.

6. The method of claim 2, wherein said plant cells comprise Taxus baccata cells.

7. The method of claim 2, wherein said gene encodes a terpene cyclase.

8. The method of claim 7, wherein said terpene cyclase is a monoterpene cyclase, diterpene cyclase, or sesquiterpene cyclase.

9. The method of claim 7, wherein said terpene cyclase is a taxadiene cyclase

10. The method of claim 2, wherein said stimulus comprises a preparation from Candida albicans.

11. The method of claim 2, wherein said stimulus comprises methyl jasmonate.

12. The method of claim 2, wherein said phenotype is the production of a catechin.

13. The method of claim 12, wherein said catechin is epi-gallocatechin gallate or epi-catechin gallate.

14. The method of claim 2, wherein said plant cells comprise cells of a species of the family Crassulaceae.

15. The method of claim 14, wherein said plant cells comprise cells of the genus Crassula.

16. The method of claim 15, wherein said plant cells comprise cells of the species C. fascicularis, C. dejecta, C. barkleyi, or C. acinaciformis.

17. The method of claim 14, wherein said plant cells comprise cells of the genus Sempervivum.

18. The method of claim 17, wherein said plant cells comprise cells of the species S. tectorum.

19. The method of claim 2, wherein said plant cells comprise cells of the family Polygonaceae.

20. The method of claim 19, wherein said plant cells comprise cells of the genus Fallopia.

21. The method of claim 20, wherein said plant cells comprise cells of the species F. convolvulus.

22. The method of claim 19, wherein said plant cells comprise cells of the genus Rumex.

23. The method of claim 22, wherein said plant cells comprise cells of the species R. obtusifolia or R. sagittatus.

24. The method of claim 2, wherein said gene associated with said desired phenotype encodes a protein that affects the accumulation of a catechin.

25. The method of claim 24, wherein said protein is a galloyltransferase, epimerase, or reductase.

26. The method of claim 2, wherein said phenotype is the accumulation of a catechin and said stimulus comprises methyl jasmonate, zeatin, 24-epibrassinolide, or 1-aminocyclopropane-1-carboxylic acid.

27. A method for producing a substantially pure catechin, said method comprising the steps of:
a) providing plant cells of the genus Crassula; and
b) purifying a catechin from said plant cells.

28. The method of claim 27, wherein said plant cells are in the form of a plant cell culture.

29. The method of claim 27, wherein said plant cells are in the form of a plant.

30. The method of claim 27, wherein said catechin is epigallocatechin gallate, epicatechin gallate, epigallocatechin, or gallocatechin.

31. The method of claim 27, wherein said plant cells comprise cells of the species C. fascicularis, C. dejecta, C. barkleyi, or C. acinaciformis.

32. A method for producing a substantially pure catechin, said method comprising the steps of:
a) providing a suspension culture of plant cells of the genus Fallopia; and
b) purifying a catechin from said plant cells.

33. The method of claim 32, wherein said catechin is epigallocatechin gallate, epicatechin gallate, epigallocatechin, or gallocatechin.

34. The method of claim 32, wherein said plant cells comprise cells of the species F. convolvulus.

35. A method for producing a substantially pure catechin, said method comprising the steps of:
a) providing a suspension culture of plant cells of the genus Rumex; and
b) purifying a catechin from said plant cells.

36. The method of claim 35, wherein said plant cells comprise cells of the species R. obtusifolia or R. sagittatus.

37. A method for identifying a compound or preparation that increases production of a catechin in a plant cell, said method comprising the steps of:
a) providing plant cells capable of producing a catechin;
b) contacting said plant cells with a candidate compound or preparation; and
c) determining the levels of said catechin in said plant cells, wherein an increase in the levels of said catechin identifies said candidate compound or preparation as a compound that increases production of said catechin or preparation.

38. The method of claim 37, wherein said plant cells are in the form of a plant cell culture.

39. The method of claim 37, wherein said plant cells are in the form of a plant.

40. The method of claim 37, wherein said catechin is epigallocatechin gallate, epicatechin gallate, epigallocatechin, or gallocatechin.

41. The method of claim 37, wherein said plant cells comprise cells from a species selected from the group consisting of C. fascicularis, C. dejecta, C. barkleyi, C. acinaciformis, F. convolvulus, R. obtusifolia, and R. sagittatus.

42. A method for identifying a protein that increases production of a catechin in a plant cell, said method comprising the steps of:
a) providing plant cells capable of producing a catechin;
b) expressing in said plant cells a nucleic acid encoding a candidate protein; and
c) determining the levels of said catechin in said plant cells, wherein an increase in the levels of said catechin identifies said candidate protein as a protein that increases production of said catechin.

43. The method of claim 42, wherein said plant cells are in the form of a plant cell culture.

44. The method of claim 42, wherein said plant cells are in the form of a plant.

45. The method of claim 42, wherein said catechin is epigallocatechin gallate, epicatechin gallate, epigallocatechin, or gallocatechin gallate.

46. The method of claim 42, wherein said plant cells comprise cells from a species selected from the group consisting of C. fascicularis, C. dejecta, C. barkleyi, C. acinaciformis, F. convolvulus, R. obtusifolia, and R. sagittatus.

47. The method of claim 42, wherein said candidate protein is a galloyltransferase, epimerase, or reductase involved in the synthesis of epigallocatechin gallate, epicatechin gallate, epigallocatechin, or gallocatechin gallate.

48. A method for functionally characterizing a protein that catalyzes the production of a catechin in a plant cell, said method comprising the steps of:
a) providing a plant cell capable of producing a precursor of said catechin but not said catechin derivative;
b) transgenically expressing in said plant cell a nucleic acid encoding a candidate protein; and
c) determining the levels of said catechin in said plant cell, wherein the production of said catechin identifies said candidate protein as a protein that catalyzes the production of said catechin.

49. The method of claim 48, wherein said plant cell is of the species C. acinaformis.

50. A method for identifying a protein that catalyzes the production of a catechin in a plant cell, said method comprising the steps of:
a) providing a first plant cell producing a precursor of said catechin but not said catechin;
b) providing a second plant cell producing said catechin;
c) identifying a transcript present in said second cell but not said first cell, wherein said transcript encodes a protein identified as one that that catalyzes the production of said catechin.

51. The method of claim 50, wherein said first plant cell is of the species C. acinaformis, and said second plant cell is of the species C. barkleyi.


SEQUENCE LISTING (15 sequences)

SEQ ID NO: 1 - protein, 216 aa, Artificial Sequence
  Synthetic
SVNDNPLIDVSRNKAIHGGNFQGTPIGVSMDNTRLALAAIGKLMFAQFSELVNDFYNNGLPSNLSGSRNPSLDYGLKGAEIAMASYCSELQFLGNPVTNHVQSAEQHNQDVNSLGLISSRKTAEAVDILKLMTSTYLVALCQAVDLRHMEENLRNTVKNTVSQVAKRTLTTGANGELHPSRFCEKDLLKVVDREYVFAYIDDPCLATYPLMQSLGA

SEQ ID NO: 2 - DNA, 648 bp, Artificial Sequence
  Synthetic
tctgtcaacgacaacccgttgatcgatgtctcgaggaacaaggccatccatggtggaaacttccaaggaaccccgatcggtgtgtccatggacaacaccaggctagcactggcagctattgggaagctcatgtttgctcagttctccgagcttgtcaatgacttctacaacaatggtctgccatcgaatctgtctggcagcaggaaccccagcttggactatgggcttaaaggagcggagatcgcaatggcttcctactgttccgarcttcagttccttggtaacccggttactaaccatgtccagagcgctgagcagcataaccaggatgtcaactcattgggattgatctcatcaaggaagacagctgaagctgttgacatcttgaagctcatgacatcgacttacttggtggccctttgccaagctgttgacctgaggcacatggaagagaatcttaggaacactgtgaagaacaccgtgagccaagtcgccaagaggacgctcacmacaggagccaacggtgagcttcacccatcgagattctgcgagaaggacttgctcaaagtrgttgacagagagtatgtgttcgcgtacattgatgacccctgcctggcaacttaccctctgatgcaaagcttaggggct

SEQ ID NO: 3 - protein, 117 aa, Artificial Sequence
  Synthetic
ENNKGARVLVICSEITAVTFRGPSDTHLYSLVGQALFGDGAAAVILGADPLPEEKPMFELVSAAQTILPDSEGAIDGHLSEVGLTFHLLKDVPGLISKNIEKGLVEAFKPIGIEDGT

SEQ ID NO: 4 - DNA, 353 bp, Artificial Sequence
  misc_feature 183: n = A,T,C or G
gagaacaacaagggcgctcgcgtgttggtgatttgctctgagatcactgctgttaccttccgtggcccaagcgatactcatttgtacagtcttgtaggtcaggccttgttcggagacggagctgcagcagtcatcctcggagcagacccccttcccgaagagaagcccatgtttgaacttgtntctgcagctcagaccatcttgccagacagtgaaggcgccatcgacggtcatcttagtgaagttggtctcacattccatttgcttaaggacgttcccgggctgatctccaagaacattgagaagggtctagtcgaggcattcaagcctatcggtatcgaagacggaactca

SEQ ID NO: 5 - protein, 122 aa, Artificial Sequence
  VARIANT 52: Xaa = Any Amino Acid
PEAVKDWREIVTYFSYPVSARDYSRWPDKPEAWKEVTKRYSDTLMGLACKLXEVLSEAMGLEKEALTKACVDMDQKVVVNYYPKCPEPDLTLGLKRHTDPGTITLLLQDQVGGLQATRDDGK

SEQ ID NO: 6 - DNA, 368 bp, Artificial Sequence
  Synthetic
cccgaggcagtgaaggaytggcgtgagattgtgacttacttctcatacccggtctcagccagggactactcacgctggccggacaagcctgaggcctggaaggaggtgaccaagcgttacagcgacacgctgatgggtctggcatgtaagcttstagaggtcttatctgaagcgatgggactagagaaggaggctctgactaaggcctgtgttgacatggaccagaaagttgttgtcaactactaccccaagtgtcctgagcctgatctaactttgggactcaagaggcataccgaccccgggacgatcaccttgcttctccaggaccaagttggcgggcttcaggccactagagatgatggtaagac

SEQ ID NO: 7 - DNA, 23 bp, Artificial Sequence
  Synthetic
gartayaayccvaagcgttttgc

SEQ ID NO: 8 - DNA, 22 bp, Artificial Sequence
  Synthetic
ggrtakatgttytcraaggcrg

SEQ ID NO: 9 - DNA, 26 bp, Artificial Sequence
  Synthetic
atgatgtaccarcargggtgcttygc

SEQ ID NO: 10 - DNA, 21 bp, Artificial Sequence
  Synthetic
agcccgggaacgtccttaagc

SEQ ID NO: 11 - DNA, 25 bp, Artificial Sequence
  Synthetic
gtsaacgacaaccckttgatcgatg

SEQ ID NO: 12 - DNA, 23 bp, Artificial Sequence
  Synthetic
acttggctcacsgtgttcttsac

SEQ ID NO: 13 - DNA, 24 bp, Artificial Sequence
  Synthetic
gaaggaggtgaccaagcgttacag

SEQ ID NO: 14 - DNA, 25 bp, Artificial Sequence
  Synthetic
tggcctgaagcccgccaacttggtc

SEQ ID NO: 15 - DNA, 18 bp, Artificial Sequence
  Synthetic
tttttttttttttttttt